(19) United States
(12) Patent Application Publication
(10) Pub. No.: US 2005/0175653 A1
(43) Pub. Date: Aug. 11, 2005

(54) Composition, process of making, uses thereof

(75) Inventors: Jean-Francois Grollier, Paris (FR); Jean Cotteret, Verneuil Sur Seine (FR); Roland De La Mettrie, Le Vesinet (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/043,285

(22) Filed: Jan. 27, 2005

Related U.S. Application Data
(60) Provisional application No. 60/545,191, filed on Feb. 18, 2004.

(30) Foreign Application Priority Data
Jan. 29, 2004 (FR) 0400855

(51) Int. Cl. A61K 7/42; A61K 7/06; A61K 35/78

(52) U.S. Cl. 424/401; 424/746; 424/769; 424/777; 424/59

(57) ABSTRACT

The invention relates to a process for preparing a composition for the cosmetic treatment of keratin materials, wherein it comprises a step of percolating a fluid at a pressure of at least 3 bar through at least one cosmetically active compound in liquid form trapped with porous or surface-treated particles.

### REFERENCE TO PRIOR APPLICATIONS

This application claims priority to U.S. provisional application 60/545,191 filed Feb. 18, 2004, and to French patent application 0400855 filed Jan. 29, 2004, both incorporated herein by reference.

### FIELD OF THE INVENTION

The present invention relates to a process for preparing a composition, preferably a cosmetic compositon, useful for example for treating human keratin materials, in particular human skin and keratin fibres such as the hair or the nails, to the composition prepared, and to a cosmetic process for treating keratin materials using this composition.

More particularly, the invention concerns a process for preparing a composition useful for example for the cosmetic treatment of keratin materials, wherein it comprises a step of percolating a fluid at a pressure of at least 3 bar through at least one cosmetically active compound in liquid form trapped with porous or surface-treated particles. The resultant composition and its uses also make up a part of the invention.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

In cosmetics, it is always sought to improve the cosmetic properties of keratin materials, for example sensitized hair, i.e. hair that has been damaged or embrittled by the chemical action of external agents, for instance atmospheric agents, seawater or swimming pool water, and/or hair treatments such as permanent-waving, relaxing, dyeing or bleaching operations. Cosmetic treatment compositions comprising cosmetically active compounds in liquid form may then be applied thereto, for instance conditioning agents, such as oils, intended to repair or limit the harmful or adverse effects produced by the various treatments or aggressions to which the keratin materials are more or less repeatedly subjected. These conditioning agents may also improve the cosmetic properties, for instance the appearance or feel of natural hair. These treatment compositions may be applied before or after the action of the atmospheric agents or treatments.

It is also known practice to use plant extracts to treat the skin, and especially to obtain softening, anti-inflammatory, anti-pruriginous, antiseptic, antiperspirant, astringent, calmative, cicatrizing, firming, fragrancing and/or tonifying properties, but also to nourish or hydrate the skin, to protect against external agents, to eliminate dead skin, to remove wrinkles and fine lines and also coloured marks (pigmentation defect or marks appearing with age, in particular on the hands, the neck or the face), or alternatively to colour the skin.

Thus, the following are used, for their texturing properties: wheat, fucus; for their antioxidant properties: rice, rosemary sage, thyme, green tea, licorice; for their foaming or emulsifying properties: soapwort, ivy, butcher's-broom, quillaja bark, quillaja, greenbrier, quinoa, soybean; for their photoprotective properties: aloe, sunflower, liquorice, magnolia, resurrection lily; for their thickening or absorbing properties: pea, wheat, potato, corn; for their odorizing properties: rosemary, violet, lavender, rose.

Various treatments of cosmetically active compounds are known.

Document U.S. Pat. No. 5,895,672 describes a system for producing tea, and especially green tea, using the Espresso technology.

Document U.S. Pat. No. 6,468,564 describes skin treatment compositions based on plant extracts.

Document U.S. Pat. No. 6,146,616 describes antioxidant compositions based on lupin oil, especially for treating the skin.

Document U.S. 2003/096038 describes a cartridge for preparing beverages. The beverage is obtained by passing pressurized water through solid compounds.

Document U.S. Pat. No. 2,526,607 describes a process for producing wax from wood, comprising the hydrolysis of the cellulose contained in the wood using an acid.

Patent application FR 2 207 699 describes a process for purifying plant extracts containing flavone pigments.

Patent application EP 0 384 796 describes a process for obtaining pyonogenol-rich extracts from plant raw material.

Patent application WO 03/095979 describes a process for treating botanical samples using pressurized steam.

Document U.S. Pat. No. 5,405,633 describes a process for extracting oils and fatty substances from natural products, using liquid propane as solvent.

Patent application EP 1 426 028 describes a process for controlling the extraction of peat using carbon dioxide.

Patent application EP 0 464 297 describes a process for preparing lipophilic plant extracts from plants in solid form.

Document U.S. 2003/157183 describes a process for encapsulating fine solid particles in the form of microcapsules.

None of these documents teaches percolating fluid through one or more cosmetically active compounds in liquid form trapped with solid particles.

However, cosmetic treatment compositions containing these cosmetically active compounds are generally aqueous compositions in which the agents must be dissolved. The lack of solubility of these compounds reduces the treating nature of these compositions. In addition, this solubility criterion reduces the number of cosmetically active compounds that may be used for the cosmetic treatment of keratin materials, and also the number of compositions containing them that may be offered to consumers. This is particularly the case for compounds with a high melting point.

Moreover, these cosmetically active compounds are generally unstable in aqueous solution and/or in atmospheric medium, or alternatively in the presence of certain chemical reagents, for instance reducing agents or oxidizing agents, which leads to (cosmetic) compositions that are relatively ineffective and/or whose appearance, odour and/or feel are unacceptable to the consumer, within a few days.

### DETAILED DESCRIPTION OF THE PREFERRED EMBODIMENTS

It has now been discovered, surprisingly, that by using a novel process for preparing a (cosmetic) composition for treating keratin materials, compositions with a higher or lower concentration of cosmetically active compound may be obtained in a very short time, for example of less than two minutes, according to need, especially free of preserving agent, making it possible to overcome the solubility problems outlined above.

This process is simple to perform and is suited to the consumer's need for an active agent or a mixture of active agents. The cosmetically active compound or mixture of compounds in liquid form is trapped with/by/in/on/etc. (hereinafter ‘trapped with’) suitable particles that are preferably porous and/or surface-treated, and a pressurized fluid, with a temperature greater than room temperature (20-25° C. at atmospheric pressure (105 Pa)) and preferably greater than 30° C., is passed for a short period, for example of less than one minute, through the cosmetically active compound or mixture of compounds trapped with these particles that are, preferably, porous or surface-treated.

The process allows the use, in anhydrous form, of cosmetically active compounds that are unstable in aqueous compositions, either by reaction with water or ambient moisture, or by reaction in aqueous solution with compounds that do not react together in anhydrous medium.

The compositions prepared according to this process may have limited stability on storage, which is not a drawback in this instance since the process leads to a ready-to-use composition, preferably intended to be used quickly after it has been prepared, for example within five minutes of its preparation, especially after cooling the composition to temperature that is acceptable for the keratin materials, especially below 60° C. and better still below 50° C. The composition may be used for up to one week or more after its preparation, depending on the rate of degradation of the cosmetically active compound or of the mixture of cosmetically active compounds.

Given the very short preparation time, the cosmetic treatment compositions may be prepared “on demand” by mixing together the various cosmetically active compounds according to the desired cosmetic properties.

According to another embodiment, the cosmetically active compound or the mixture of cosmetically active compounds and/or cationic conditioning agents may be packaged in a ready-to-use device, and it is not necessary to determine beforehand the concentrations of active compounds in solution, which limits the measuring errors by the user.

In addition, the process according to the invention makes it possible to avoid the use of multi-compartment bottles, which makes the process particularly economical and safer for the user.

The composition thus obtained may be used alone or as a mixture with another composition.

A further advantage of this preparation process is the production of compositions that give good cosmetic properties.

One subject of the invention is thus a process for preparing a (cosmetic) composition for treating keratin materials, comprising a step of percolating a fluid at a pressure of at least 3 bar through at least one cosmetically active compound trapped with solid particles that are especially porous or surface-treated.

Another subject of the invention is a composition obtained via the process according to the invention.

A subject of the invention is also the use, especially the cosmetic use, of the composition obtained according to the process of the invention, for example as an agent for the cosmetic treatment of keratin materials.

Another subject of the invention is a packaging device for performing the preparation process of the present invention.

A subject of the invention is also a (cosmetic) process for treating human keratin materials, comprising a) the preparation of a ready-to-use (cosmetic) composition by percolating a fluid at a pressure of at least 3 bar through at least one cosmetically active compound trapped with porous or surface-treated solid particles, and b) the application of the composition obtained in step a) to the keratin materials.

Depending on the nature of the active agents, the application to the keratin materials may be performed topically or orally, and preferably topically.

Other subjects, characteristics, aspects and advantages of the invention will emerge even more clearly on reading the further nonlimiting description and the examples that follow.

The term “cosmetically active compound” means any compound with cosmetic or dermatological activity, i.e. a compound that has beneficial action on the skin or keratin fibres.

According to the invention, the process for preparing a (cosmetic) composition for treating keratin materials comprises a step of percolating a fluid preferably at a temperature at least equal to 30° C., for example between 30° C. and 150° C. and better still ranging from 40 to 120° C., at a pressure of at least 3 bar (3×105 Pa), through at least one cosmetically active liquid compound trapped with particles that are preferably porous or surface-treated solid particles.

The notions “liquid” and “solid” refer to a physical state of an active compound or of particles at a temperature of 20-25° C. and an atmospheric pressure of 105 Pa. In addition, the particles advantageously have a high melting point, i.e. at least higher than the percolation temperature used.

Percolation is a movement of fluid through a saturated porous medium, allowing the passage of the fluid due to the action or effect of pressure.

The fluid may comprise steam optionally accompanied by liquid water, and/or one or more cosmetically acceptable, especially organic, liquid and/or gaseous solvents. Preferably, the fluid comprises at least steam possibly accompanied by liquid water, and even more preferably it is steam possibly accompanied by liquid water.

Examples of organic solvents that may be mentioned include C1-C4 lower alcohols, such as ethanol and isopropanol; polyols and polyol ethers, for instance 2-butoxyethanol, propylene glycol, propylene glycol monomethyl ether, diethylene glycol monomethyl ether and monoethyl ether, and also aromatic alcohols, for instance benzyl alcohol or phenoxyethanol, and mixtures thereof.

The term “water-insoluble compound” means any compound which, at a concentration of greater than or equal to 0.1% by weight in water at 25° C., does not form an isotropic solution that is transparent to the naked eye.

The cosmetically active compound in liquid form is trapped with solid particles or immobilized on solid particles, these particles being capable of adsorbing or absorbing the compound; these particles are especially porous or surface-treated. The term “surface-treated particles” refers to particles that have been treated so as to be able to attach the cosmetically active compound, for example by adsorption.

The trapping of the cosmetically active compound in liquid form may take place by placing the cosmetically active compound in contact with the porous or surface-treated solid particles, by means of numerous techniques known to those skilled in the art. Among these techniques that may be mentioned are impregnation, freeze-drying, described especially in patent FR 2 099 049, spray-drying and atomization.

The term “keratin materials” means the skin, the scalp and the lips, and/or integuments such as the nails, the eyelashes, the eyebrows and the hair.

The process of the present invention may be performed using a standard device for generating a pressurized fluid, at a temperature above room temperature, preferably at least equal to 30° C., for example between 30° C. and 150° C. and even more preferably ranging from 40° C. to 120° C. Such a device comprises a pressure-resistant chamber, equipped with a heating unit, and also a circuit for conveying the fluid produced to the trapped cosmetically active compound.

According to another embodiment, the device further comprises a reservoir of liquid(s) and also a pump for conveying the liquid(s) to the chamber.

The liquid contained in the reservoir is preferably either water, or a cosmetically acceptable solvent or a mixture of several cosmetically acceptable solvents, or alternatively a mixture of water and of one or more cosmetically acceptable solvents. Preferably, the liquid comprises at least water, and even more preferably it is water.

A device that is particularly useful for performing the process of the present invention is a coffee machine of the “espresso” type. Such machines are well known in the art. For example, these machines are described in patents AT 168 405, U.S. Pat. No. 2,688,911, DE 324 33 870 and IT 1 265 636.

According to one particular embodiment of the invention, the percolation step is performed with a fluid at a temperature of greater than or equal to 30° C., especially ranging from 30° C. to 150° C. and preferably ranging from 40° C. to 120° C., at a pressure of between 3 and 30 bar, or of at least 4 bar, preferably greater than 10 bar and most particularly between 10 and 30 bar.

The cosmetically active compound(s) trapped with solid particles (or immobilized on these particles) may be used directly, in the device for generating the pressurized fluid, in a container designed for this purpose. They may also be packaged in a particular device for packaging a (cosmetic) composition, comprising a closed housing delimited by at least one wall that is at least partially permeable to a fluid under a pressure of at least 3 bar, the composition containing at least one cationic conditioning agent, in solid or pasty form. Such devices are described, for example, in patent applications WO 00/56629, EP 512 470, U.S. Pat. No. 5,897,899 or WO 99/03753. These packaging devices are generally airproof, waterproof and/or lightproof.

According to one particular embodiment, the housing is delimited by two sealed sheets. According to another embodiment, the housing is delimited by a tray closed with a lid.

These devices may be manufactured from woven or nonwoven plastic or plant materials, for example cellulose, metal such as aluminium, or composite materials. Such devices are described, for example, in patent applications WO 00/56629, EP 512 470, U.S. Pat. No. 5,897,899 or WO 99/03753.

The cosmetically active compound in liquid form or the mixture of cosmetically active compounds may be chosen from aqueous or aqueous-alcoholic extracts that may be trapped or immobilized on solid particles, especially porous or surface-treated solid particles.

The porous or surface-treated solid particles are in particular chemically inert (i.e. they do not react chemically with the active agent(s) they are trapping); they may be mineral or organic, lamellar, spherical or oblong, or any shape. They are chosen, for example, from alumina, zeolite, chitosan, maltodextrin, cyclodextrin, carrageenans, zinc oxide or zirconium oxide, silica, talc, precipitated calcium carbonate and magnesium carbonate or magnesium hydroxycarbonate particles, hollow resin particles, especially silicone or polysiloxane particles, hollow silica microspheres (Silica Beads® from Maprecos), cotton wool, superabsorbent compounds, starch, which is optionally modified, corn, wheat, rice, barley or pea grain or flour, clay, sawdust, calcium alginate or oligosaccharide alginate, active charcoal, hollow poly(vinylidene chloride/acrylonitrile) particles, especially those sold under the general name “Expancel®” by the company Akzo Nobel under the specific references “Expancel® WE” or “DE” Expancels, hollow polyamides and especially Nylon powders (for instance Orgasol® from Atochem), porous ceramic particles, porous hydroxyapatite and hollow glass particles, and mixtures thereof.

Preferably, Nylon, porous silica, maltodextrin, starch, carrageenans, Expancel, calcium alginate, oligosaccharide alginate or polysiloxane particles are used.

The cosmetically active compound(s) in liquid form may preferably be chosen from:
aqueous, aqueous-alcoholic or oily extracts of plants, for instance extracts of witch hazel for astringent and fragrancing properties, glycolic extracts of lily flower (fragrancing active agent), extract of Iris florentina (anti-ageing active agent), extract of salad burnet for treating irritated skin (for example skin irritated by shaving or by sunlight), fruit acids (glycolic acid, lactic acid) as a desquamating agent, aqueous extracts of Iris pallida (anti-irritant), the aqueous-glycolic extract of sage (as a cicatrizing, anti-inflammatory or slimming active agent), and extract of tomato (especially lycopene) as an antioxidant;
unsaturated essential fatty acids (for instance linoleic acid or linolenic acid), as an emollient active agent, or unsaturated essential fatty alcohols (for instance oleyl alcohol or linolenyl alcohol) with emollient action;
non-volatile oils, for instance unsaturated plant oils generally containing unsaturated esters (soybean oil, olive oil, grapeseed oil or coriander oil) with emollient properties and, for coriander, anti-inflammatory properties;
fragrances (known for their instability to light) and essential oils;
aqueous hydrogen peroxide solution;
reducing agents in liquid form, for instance thiolactic acid and its esters (especially glyceryl monothiolactate), thioglycolic acid and its esters, especially glyceryl or glycol monothioglycolate, diammonium dithiodiglycolate or ammonium thioglycolate, and thioglycerol;
sunscreens in liquid form, for instance Cinoxate, ethylhexyl dimethyl PABA, ethylhexyl methoxycinnamate, Homosalate, menthyl anthranilate, Octocrylene, ethylhexyl salicylate and PEG-25 PABA;
vitamins or vitamin derivatives in liquid form, for instance vitamin A and its esters such as vitamin A palmitate, and vitamin E and its esters, for instance vitamin E acetate or palmitate;
liquid esters of monoacids or polyacids with monoalcohols or polyols, such as isopropyl myristate, octyl isononanoate or tridecyl isononanoate;
silicone oils such as volatile liquid polydimethylsiloxanes and cyclomethicones, the temperature of the percolator however needing to be about 30° C. and preferably between 30 and 40° C. for the oils;
liquid surfactants, for instance liquid oxyethylenated fatty alcohols such as laureth-2 and oxyethylenated sorbitan monolinoleate or trioleate.

The non-volatile oils mentioned above may be chosen from those conventionally used in cosmetics. The term “oil” means any fatty substance that is liquid at 25° C., at 1 atm, and having a molecular weight of greater than or equal to 160 and especially between 170 and 106, or even between 200 and 5×105, which is compatible with application to the skin, the lips and/or the integuments (nails, eyelashes, eyebrows or hair).

The oils that may be used in the process according to the invention may be of animal, plant, mineral or synthetic origin. They generally serve to condition the hair, and to protect and nourish the skin and/or afford it emollience.

As oils other than those mentioned above, mention may especially be made of:
oils of plants or animal origin such as sunflower oil, evening primrose oil, safflower oil, passionflower oil, rye oil, marrow oil, sesame seed oil, hazelnut oil, apricot kernel oil, almond oil, avocado oil, beauty-leaf oil, macadamia oil, grapeseed oil, coconut oil, groundnut oil, almond oil, castor oil, jojoba oil or cereal germ oils such as wheat germ oil or corn germ oil, shea butter oil; fish oils, perhydro squalene, glyceryl tricaprocaprylate;
synthetic oils, for instance the oils of formula R1COOR2 in which R1 represents a higher fatty acid residue containing from 7 to 29 carbon atoms and R2 represents a hydrocarbon-based chain containing from 3 to 30 carbon atoms, for instance purcellin oil, butyl myristate, isopropyl myristate, cetyl myristate, isopropyl palmitate, 2-ethylhexyl palmitate, isopropyl adipate, ethylhexyl adipate, butyl stearate, hexadecyl stearate, isopropyl stearate, octyl stearate, isocetyl stearate, 2-octyldodecyl stearate, decyl oleate, hexyl laurate, 2-octyldodecyl erucate, isononyl isononanoate and isostearyl isostearate; hydroxylated esters, for instance isostearyl lactate, octyl hydroxystearate, octyldodecyl hydroxystearate, diisostearyl malate and triisocetyl citrate; heptanoates, octanoates or decanoates of fatty alcohols; polyol esters, for instance glyceryl or ethylene glycol octanoate or decanoate, propylene glycol dicaprylate, propylene glycol dioctanoate, neopentyl glycol diheptanoate or diethylene glycol diisononanoate; pentaerythritol esters, for instance pentaerythrityl tetraisostearate; esters of the type such as tridecyl trimellitate;
linear or branched hydrocarbons of mineral or synthetic origin, such as liquid paraffins and derivatives thereof, poly(α-olefins), petroleum jelly, polydecenes, and hydrogenated polyisobutenes such as parleam;
glycerides, especially acetylglycerides or fatty acid triglycerides containing 4 to 10 carbon atoms, for instance heptanoic, octanoic and capric/caprylic acid triglycerides, and saturated C10-18 fatty acid triglycerides;
fluoro oils and perfluoro oils, lanolin, hydrogenated lanolin and acetylated lanolin.

Preferably, the cosmetically active liquid active agents that may be used in the invention are essential oils, liquid plant extracts and especially extracts of Iris pallida, of tomato (lycopene) or of grape-cake fruit acids, extract of aloe, DHA, screening agents, liquid vitamins and liquid esters thereof, and esters of monoacids and of monoalcohols.

It is possible to use dispersions of solid compounds in these liquid compounds and thus to be able to absorb them onto or to absorb them in an optionally porous solid support.

It is possible for the user to use cosmetically active compounds that are sold already trapped on absorbent and especially porous particles. Examples that may be mentioned include the extract of balm atomized onto maltodextrin particles; liquid α-hydroxy acids atomized onto a polysaccharide and/or starch particles (50/50 powdered glucuronic/mannuronic acids atomized onto oligosaccharide alginate particles, sold and/or manufactured by the company Codif for soothing and/or anti-inflammatory properties); polyamides-12 particles impregnated with dihydroxyacetone (DHA) (sold and/or manufactured under the name Orgasol DHA or Orgasol 2002 N5 HA by the company Atochem) and carrageenan microspheres impregnated with DHA, as an oily dispersion (sold and/or manufactured under the name DHA 2011 by the company Lipotec) for colouring the skin; aloe sap or exudate for soothing, anti-irritant properties on the skin or for hair beauty, trapped on agar spheres (Softsphere SP-VEAAV GRP 41 M from Kobo); powdered concentrated red elder sap, atomized onto maltodextrin (elder powder from the company Diana Vegetal); extract of grape-cake on maltodextrin, as a free-radical scavenger or antioxidant (for example the grape-cake polyphenols sold under the name ACWS P 118 by the company Sefcal); polysiloxane nanospheres filled with vitamin A palmitate or polyamides-12 microspheres impregnated with vitamin A palmitate (Orgasol 2002 UD+10% vitamin A from Atochem) for its nourishing or anti-inflammatory properties, and porous silica microbeads filled with vitamin E acetate (SB-700/VEAC from Myoshi Kasei) for its moisturizing and antioxidant properties.

The trapped cosmetically active compound(s) according to the invention may be used as a mixture with one or more solid or pasty and preferably pulverulent adjuvants. The adjuvants may be chosen from clays, salts, anionic, nonionic, amphoteric or zwitterionic surfactants, natural or synthetic thickeners, glass beads, silica, Nylon, alumina, titanium dioxide, zeolites, polymethyl methacrylate (PMMA), chitosan, maltodextrin, cyclodextrin, mono- or disaccharides, for instance glucose, sucrose, sorbitol or fructose, zinc oxide, zirconium oxide, resin particles, for instance silicone or silica beads, talc, polyaspartic acid, borosilicates, especially calcium borosilicate, polyethylene, cotton, polytetrafluoroethylene (PTFE), cellulose and its derivatives, superabsorbent compounds, magnesium carbonate, calcium carbonate, corn seeds, polydimethylsiloxane gums, polyacrylamide, porous hydroxyapatite, silk, collagen, sawdust, wrack powder, meals or extracts of wheat, rice, pea, lupin, soybean or barley, crosslinked polyvinylpyrrolidone, calcium alginate, active charcoal, and poly(vinylidene chloride/acrylonitrile) particles, especially those sold under the general name “Expancel®” by the company Akzo Nobel under the particular reference “Expancel® WE” or “Expancel® DE”, and mixtures thereof.

Preferably, as solid or pasty adjuvants that may be used in the invention, mention may be made of silica, Expancel, Nylon, PTFE, cellulose or starch. When one or more adjuvants are present, the trapped cosmetically active compound(s) of the invention is (are) present in the composition subjected to the percolation under pressure preferably in an amount ranging from 0.5% to 99% by weight, better still from 1% to 80% by weight and even more preferably from 2% to 60% by weight relative to the total weight of trapped cosmetically active compound(s) and adjuvants.

The plant or plant extracts used may be subjected, prior to percolation, to one or more treatments such as roasting, cryogrinding or lyophilization.

The composition obtained according to the process of the invention comprises, besides the trapped cosmetically active compound(s) and the component(s) of the fluid, i.e. water and/or the cosmetically acceptable solvent(s), optionally all or some of the adjuvants(s) present in the solid or pasty mixture, depending on the chemical nature of these adjuvants, and especially their ability to be entrained by the pressurized fluid, and their size.

The invention also relates to a composition obtained via the process according to the invention, the composition preferably being free of preserving agents.

Using the preparation process of the invention, a (cosmetic) composition for treating keratin materials is obtained, which may be applied directly to keratin materials, or which may be mixed with a cosmetically acceptable medium, or alternatively at least one additive conventionally used in cosmetics may be added thereto by an operator. At least two compositions obtained via the process of the invention may also be mixed together. The (cosmetic) composition for treating keratin materials optionally resulting from the mixture(s) and/or addition(s) indicated above will be referred to hereinbelow as the final (cosmetic) treatment composition or final composition.

One particular mode of implementation of the invention consists in applying the composition obtained by means of a device not requiring any human intervention, and optionally equipped with a cooling means.

The amount of the trapped cosmetically active compound(s) present in the final (cosmetic) treatment composition obtained via the process of the present invention is generally preferably between 0.001% and 50% by weight approximately, preferably between 0.005% and 30% and even more preferably between 0.01% and 20%, relative to the total weight of the final (cosmetic) treatment composition.

When the (cosmetic) composition obtained via the percolation process of the present invention is mixed with a cosmetically acceptable medium, the medium generally comprises water or a mixture of water and of at least one organic solvent to dissolve the compounds that would not be sufficiently soluble in water. The term “cosmetically acceptable” means a medium that is compatible with keratin materials and especially the skin, the lips and/or the integuments, and that also has an appearance, a field, an odour and, possibly, a taste that the user finds pleasant.

Examples of organic solvents that may be mentioned include C₁-C₄ lower alcohols, such as ethanol and isopropanol; polyols and polyol ethers, for instance 2-butoxyethanol, propylene glycol, propylene glycol monomethyl ether and diethylene glycol monomethyl ether and monoethyl ether, and also aromatic alcohols, for instance benzyl alcohol or phenoxyethanol, and mixtures thereof.

The solvents are preferably present in proportions preferably of between 1% and 40% by weight and even more preferably between 5% and 30% by weight relative to the total weight of the final composition.

At least one additive conventionally used in cosmetics may also be added to the cosmetic treatment compositions obtained according to the process of the present invention. Examples of such additives that may be mentioned include anionic, nonionic, amphoteric or zwitterionic surfactants, or mixtures thereof, anionic, nonionic, amphoteric or zwitterionic polymers, or mixtures thereof, mineral or organic thickeners, and in particular anionic, cationic, nonionic and amphoteric polymeric associative thickeners, antioxidants, penetrating agents, sequestering agents, fragrances, buffers, dispersants, conditioning agents other than those described above, for instance non-cationic silicone oils, film-forming agents, ceramides, preserving agents and opacifiers, and also oils, waxes, gums and coloured or nacreous pigments.

The above additives are generally present in an amount for each of them of between 0.01% and 20% by weight relative to the weight of the final composition.

Needless to say, a person skilled in the art will take care to select this or these optional compounds such that the advantageous properties intrinsically associated with the (cosmetic) composition in accordance with the invention are not, or are not substantially, adversely affected by the addition(s) envisaged.

The pH of the final composition is generally between 3 and 12 and preferably between 5 and 11. It may be adjusted to the desired value using acidifying or basifying agents usually used in cosmetics, or alternatively using standard buffer systems.

Among the acidifying agents that may be mentioned, for example, are mineral or organic acids such as hydrochloric acid, orthophosphoric acid, sulfuric acid, carboxylic acids such as acetic acid, tartaric acid, citric acid and lactic acid, and sulfonic acids.

Among the basifying agents that may be mentioned, for example, are aqueous ammonia, alkaline carbonates, alkanolamines such as mono-, di- and triethanolamine and derivatives thereof, sodium hydroxide, potassium hydroxide and the compounds of formula (II) below: 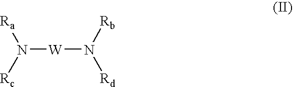
in which W is a propylene residue that is optionally substituted with a hydroxyl group or a C1-C4 alkyl radical; Ra, Rb, Rc and Rd, which may be identical or different, represent a hydrogen atom, a C1-C4 alkyl radical or a C1-C4 hydroxyalkyl radical.

The final (cosmetic) treatment composition may be in various forms, such as in the form of liquids, creams, gels or mascara, or in any other form that is suitable for treating keratin materials, and especially keratin fibres, and the skin. This final composition may be used for treating and/or caring for the hair and/or for treating and/or caring for and/or making up the skin, the nails, the eyelashes or the lips.

The final (cosmetic) treatment composition may be used, for example, as a shampoo, a rinse-out or leave-in conditioner, a deep-down care mask, a shower gel, or a lotion or cream for treating keratin materials.

The present invention also relates to a cosmetic process for treating keratin materials, comprising the preparation of a cosmetic treatment composition according to the process as defined above, and its application to the keratin materials, for example by means of an operator or by means of a device not requiring any human intervention. The application time may range for example between 15 seconds and one hour.

Before application, the cosmetic treatment composition obtained according to the process of the invention may be mixed with a cosmetically acceptable medium and/or with one or more additives conventionally used in cosmetics, as described above.

Another mode of implementation comprises preparing at least two cosmetic treatment compositions according to the process of the invention, mixing them together, and optionally adding a cosmetically acceptable medium and/or one or more additives conventionally used in cosmetics, as described above, and then in applying the final composition obtained to keratin materials.

The total amount of the trapped cosmetically active compound(s) on solid particles, optionally combined with one or more pasty or solid adjuvants, depends on the size of the percolation chamber or on the leaktight packaging used, and also on the density of the ingredients placed in the percolation chamber or in the single-dose packaging device. In practice, this amount may range for example from 1 g to 10 g, more particularly from 2 to 5 g.

The example below is intended to illustrate the present invention.

### EXAMPLE 1

In this example, a commercial Espresso machine with a chamber volume of 13 ml is used.

5 g of powdered carrageenan microspheres impregnated with dihydroxyacetone in oily dispersion, sold under the name DHA 2011 by the company Lipotec are prepared.

This mixture is placed in the commercial Espresso machine as described in documents mentioned above. Steam is then passed through until a composition (A) with a final volume of 50 ml is obtained.

One part by weight of an aqueous composition (B) containing 1% by weight of hydroxyethylcellulose is then added to two parts by weight of composition (A).

A dye composition ready to be applied to the skin is thus obtained.

### EXAMPLE 2

5 g of porous silica microbeads filled with vitamin E acetate and 4 g of ammonium bicarbonate are placed in a commercial Espresso machine. Steam is then passed through until a composition with a final volume of 50 ml is obtained.

A hair-conditioning composition ready to be applied to the hair to nourish and condition it is thus obtained.

The above written description of the invention provides a manner and process of making and using it such that any person skilled in this art is enabled to make and use the same, this enablement being provided in particular for the subject matter of the appended claims, which make up a part of the original description and including a process for preparing a (cosmetic) composition useful for treating keratin materials, wherein it comprises a step of percolating a fluid under a pressure of at least 3 bars through at least one one cosmetically active compound in liquid form trapped with solid particles, the composition prepared, and a process using the composition for the permanent or semi-permanent coloring of the skin, the eyelids, the lips or the nails, for example.

As used herein, the phrases “selected from the group consisting of,” “chosen from,” “selected from,” and the like include mixtures of the specified materials.

Where compounds are described as, e.g, “cellulose and its derivatives” and the like an alternate is “cellulose and cellulose compounds” where cellulose derivatives and compounds share a common core/structure with cellulose. One of ordinary skill in the art knows how to identify derivatives and compounds of compound X based on the structure of X and the similarity in structure of the derivatives and compounds.

Where a term is enclosed by parentheses it is an optional modifier. For example, the term “(cosmetic) composition” describes both a composition in general and a cosmetic composition.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for preparing a composition, comprising percolating a fluid at a pressure of at least 3 bar through at least one cosmetically active compound in liquid form trapped with solid particles.

2. A process according to claim 1, wherein the solid particles are porous and/or surface-treated.

3. A process according to claim 1, wherein the at least one cosmetically active compound in liquid form trapped with solid particles are prepared by placing the cosmetically active compound(s) in liquid form in contact with the particles.

4. A process according to claim 3, wherein the trapping is performed by at least one of impregnation, freeze-drying, spray-drying and atomization.

5. A process according to claim 1, wherein the solid particles are chosen from alumina, zeolite, chitosan, maltodextrin, cyclodextrin, carrageenans, zinc oxide or zirconium oxide, silica, talc, precipitated calcium carbonate and magnesium carbonate or magnesium hydroxycarbonate particles, hollow resin particles, hollow silica, cotton wool, superabsorbent compounds, starch, which is optionally modified, corn, wheat, rice, barley or pea grain or flour, clay, sawdust, calcium alginate or oligosaccharide alginate, active charcoal, poly(vinylidene chloride/acrylonitrile) particles, hollow polyamide powders, porous ceramic particles, porous hydroxyapatite and hollow glass particles, and mixtures thereof.

6. A process according to claim 1, wherein the cosmetically active compound(s) is (are) chosen from plant extracts, unsaturated essential fatty acids, non-volatile oils, fragrances, essential oils, aqueous hydrogen peroxide solution, reducing agents in liquid form, sunscreens in liquid form, vitamins or vitamin derivatives in liquid form, liquid esters of monoacids or of polyacids with monoalcohols or polyalcohols, silicone oils, liquid surfactants and mixtures thereof.

7. A process according to claim 6, wherein the cosmetically active compound(s) is (are) chosen from extracts of witch hazel, glycolic extracts of lily flower, extract of Iris florentina, extract of salad bumet, fruit acids, aqueous extracts of Iris pallida, aqueous-glycolic extracts of sage, extract of tomato (lycopene), and mixtures thereof.

8. A process according to claim 1, wherein the trapped cosmetically active compound(s) is (are) present as a mixture with at least one adjuvant.

9. A process according to claim 8, wherein the adjuvant is chosen from clays, salts, anionic, nonionic, amphoteric or zwitterionic surfactants, natural or synthetic thickeners, glass beads, silica, Nylon, alumina, titanium dioxide, zeolites, poly(methyl methacrylate) (PMMA), chitosan, maltodextrin, cyclodextrin, mono- or disaccharides, zinc oxide, zirconium oxide, resin particles, talc, polyaspartic acid, borosilicates, polyethylene, cotton, polytetrafluoroethylene (PTFE), cellulose and its esters, superabsorbent compounds, magnesium carbonate, calcium carbonate, corn, wheat, rice, pea, lupin, soybean or barley grain or flour, polydimethylsiloxane gums, polyacrylamide, silk, collagen, sawdust, fucus powder, crosslinked polyvinylpyrrolidone, calcium alginate, active charcoal and poly(vinylidene chloride/acrylonitrile) particles, and mixtures thereof.

10. A process according to claim 8, wherein the trapped cosmetically active compound(s) is (are) present in an amount ranging from 0.5% to 99% by weight relative to the total weight of trapped cosmetically active compound(s) and adjuvant.

11. A process according to claim 1, wherein the percolation step is performed with a fluid at a pressure of between 3 and 30 bar.

12. A process according to claim 1, wherein the percolation step is performed with a fluid at a pressure of at least 10 bar.

13. A composition prepared by a process that comprises percolating a fluid at a pressure of at least 3 bar through at least one cosmetically active compound in liquid form trapped with solid particles.

14. The composition according to claim 13, wherein said composition is free of preserving agents.

15. A process for treating keratin materials, comprising applying a composition according to claim 13 to keratin material.

16. The process according to claim 15, wherein the composition is applied to the keratin material by a device not requiring any human intervention.

17. The process according to claim 15, wherein, before the application, the composition is mixed with a cosmetically acceptable medium and/or with one or more cosmetic additives, and/or with another composition prepared by a process that comprises percolating a fluid at a pressure of at least 3 bar through at least one cosmetically active compound in liquid form trapped with solid particles.

18. A device, comprising a closed housing delimited by at least one wall that is at least partially permeable to a fluid at a pressure of at least 3 bar, the housing containing at least one cosmetically active compound in liquid form trapped with particles.

19. The device according to claim 18, wherein the housing is delimited by two sealed sheets.

20. The device according to claim 18, wherein the housing is delimited by a tray closed with a lid.
